(19) United States
(12) Patent Application Publication
(10) Pub. No.: US 2008/0275493 A1
(43) Pub. Date: Nov. 6, 2008

(54) EXTENDED DURATION MEDICAL FILTER SYSTEM WITH CAGED FILTER

(76) Inventor: Victor Farmiga, Putnam Valley, NY (US)

Correspondence Address: RONALD L. HOFER, 122 LINDBERGH LANE, MOORESVILLE, NC, 28117, US

(21) Appl. No.: 11/742,746

(22) Filed: May 1, 2007

(51) Int. Cl. A61M 29/00

(52) U.S. Cl. 606/200

(57) ABSTRACT

A removable, radially compressible and expandable medical filter system for placement in a tubular body passage such as a blood vessel. The medical filter system comprises in radially expanded form, an elongated tubular cage with a hollow interior and an elongated filter disposed within the hollow interior of the cage in an interference fit.

### BACKGROUND AND SUMMARY OF THE INVENTION

The present invention relates to a medical filter system which can be placed inside a blood vessel or other body passage. More specifically, the present invention relates to a medical filter system which includes a cage which contains a medical filter and which is intended for placement in a blood vessel or other body passage.

Medical filters, such as vena cava filters, are emplaced inside blood vessels or other body passages for the purpose of intercepting thrombus or particles while allowing free flow of blood in the vessels or other body fluid in the body passages. Medical filters often are emplaced and never retrieved, remaining effective during their time in place and remaining permanently in place. However, while a medical filter may be implanted permanently, it may be desirable to retrieve the filter. If retrieval is desired, it should be retrieved as easily as possible with minimal trauma to the vessel or body passage. If the filter has just been implanted, retrieval may be relatively easy. But in some cases it may be desirable to remove or retrieve a medical filter after it has been in place for an extended period of time, for example, after it has been in place for more than two weeks.

Generally speaking, it is known to emplace or deliver medical filters in body passages as well as to remove the medical filters following their implantation. For example, it is known to implant and retrieve vena cava filters in blood vessels although retrieval of vena cava filters after they have been in place for an extended period of time can be challenging. Vena cava filters commonly have a network of interconnected elements and after implantation, self-expand radially in the blood vessel to a size matching the anatomy of the associated blood vessel. Vena cava filters commonly anchor themselves in the blood vessel to resist migration therein and have a filter structure which is in contacting relationship with the wall of the blood vessel. However, immediately after being implanted in the blood vessel, there is a proliferation of tissue cell growth of the vessel which leads to endothelialization or growth of cellular structure of the vessel around part of the filter structure. This growth can makes it challenging to remove the filter without trauma to the wall of the vessel.

In light of the challenge posed by removal of an extended duration medical filter, it would be beneficial to have an improved, extended duration medical filter system designed to be permanently in place but readily removed with minimal trauma if such removal is desired. It would also be desirable to have a practical and economical medical filter system. Furthermore, it would be desirable to have a medical filter system which can be implanted and removed from either a femoral or jugular approach and which can be adapted for use with a wide variety of specific filter basket designs. It also would be desirable to have a medical filter system which ensured optimal orientation of the filter in a vessel and to have a filter system which was capable of serving more than one purpose, i.e., could provide a reinforcement of a vessel wall while also providing a filter function.

Accordingly, the present invention provides an improved medical filter system for placement in a tubular body passage such as a blood vessel. The system is radially compressible and expandable and includes a filter which is removable, the removable feature being enhanced by the filter system design.

A medical filter system of the present invention comprises two parts: a cage and a filter, each of which is self-expanding. The cage is a tubular stent or graft adapted for placement in a vein or other body passage. The cage has a hollow interior and

Thus, in accordance with the present invention, after a filter of this invention has been implanted in a vessel or the like, it is contemplated that the bio-absorbable barbs will initially secure the filter structure to the vessel and will stand-off the main structure of the filter from the vessel wall during the initial proliferation of tissue re-growth occurring after its implantation in the vessel. Then the barbs will be bio-absorbed, leaving the main structure secured in the vessel but with an amount of tissue over the structure allowing its removal from the vessel.

In accordance with the method of the present invention, a medical filter of the present invention is implanted in a body passage, the barbs are bio-absorbed therein and then the filter is removed therefrom by using a retrieval catheter.

Further understanding of the present invention will be had from the following description taken in conjunction with the accompanying drawings and appended claims.

### BRIEF DESCRIPTION OF THE DRAWINGS

FIG. 1 is a perspective view, showing a preferred embodiment of a medical filter system of the present invention;

FIG. 2 is a side elevation, showing in separate and expanded form, the cage and filter of the preferred embodiment of FIG. 1;

FIG. 3 is a longitudinal sectional view showing the preferred embodiment of FIG. 1 in a blood vessel; and

FIG. 4 is longitudinal sectional view illustrating a step in the retrieval of the filter of the preferred embodiment of FIG. 1.

### DETAILED DESCRIPTION OF THE INVENTION

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the present invention may be used in veins but is also useful in other body passages. Also, while the preferred embodiment of the present invention is described herein as a specific, preferred cage and filter structure, it will be appreciated by those skilled in the art that other designs of cages and filter structures are suitable and come within the spirit and broad scope of this invention.

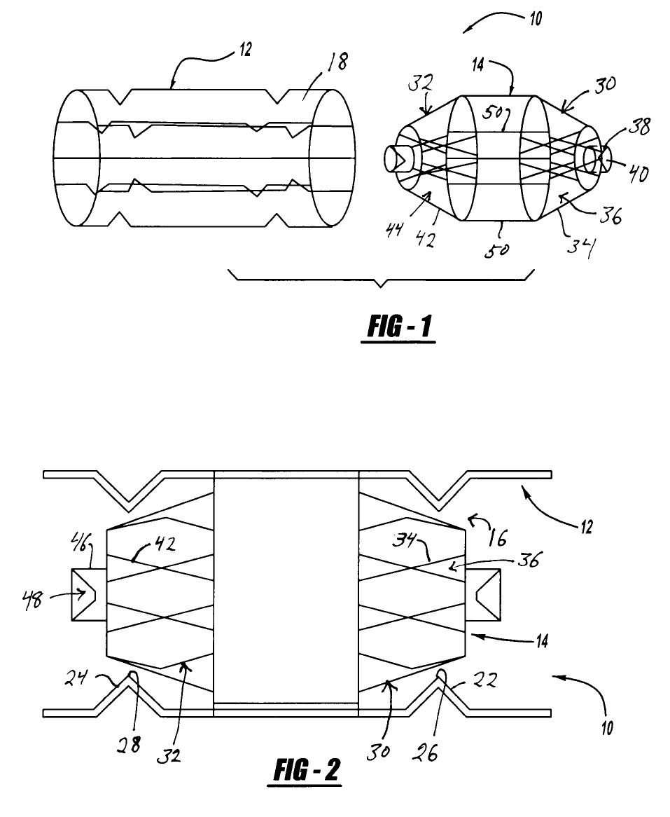

Now referring to FIG. 1, a preferred embodiment of a medical filter system of the present invention is shown and indicated generally by the numeral 10. FIG. 1 shows medical filter system 10 in radially expanded form. Broadly speaking, medical filter system 10 comprises a cage 12 and a filter 14. As will be described in more detail below, cage 12 has a hollow interior 16 which holds filter 14 and prevents unwanted axial movement of filter 14.

Cage 12 is shown in the form of a tube or stent comprised of a wire metal mesh material 18 in the form of an expanded tube. Cage 12 is radially compressible and expandable and may be self-expanding or expandable by means of a balloon catheter or the like. Thus, it is contemplated that cage 12 can be made of a resilient material having a memory such that it can be formed into a pre-selected, expanded shape and radially compressed to be inserted into the lumen of a delivery catheter for implantation in a body passage such as a blood vessel whereupon it will expand to the size of the body passage. Alternatively, cage 12 can be made of a material that is compressible for insertion into a delivery catheter and then expandable after delivery to the desired site in the body passage by means of a balloon catheter or the like.

As best shown in FIG. 2, cage 12 has a generally tubular shape with a hollow interior 16. Hollow interior 16 has an interior diameter 20 for receipt of filter 14 and further has circumferential, radially inwardly extending ribs 22 and 24 which provide reduced interior diameters 26 and 28. Ribs 22 and 24 serve to interfere with axial movement of filter 14 after filter 14 has expanded within hollow interior 16 of cage 12.

Filter 14 has first and second filter baskets 30 and 32. Filter basket 30 has a plurality of filter elements 34 which form diamond shaped openings 36. The radially inward end of each element 34 is connected to first central member 38 which carries retrieval hook 40. Filter basket 32 has a plurality of filter elements 42 which form diamond shaped openings 44. The radially inward end of each filter element 42 is connected to second central member 46 which has a retrieval hook 48. A plurality of struts 50 extend between filter basket 30 and filter basket 32, each strut 50 being connected to a radially outward end of a filter element 34 and a radially outward end of a filter element 42.

It will be appreciated by those skilled in the art that the structure of filter 14 is generally that disclosed in U.S. Pat. No. 6,989,021 B2, Jan. 24, 2006 to Bosma et al. for “Retrievable Medical Filter,” the disclosure of which is specifically incorporated by reference herein. Of course, the anchoring ski barbs shown in the Bosna et al. patent (as anchoring barbs 26 therein) are not required in the present invention which relies on cage 12 for anchoring. It will also be appreciated by those skilled in the art that, while filter 14 is a preferred filter for use in the filter system 10 of the present invention, that other designs of filters, both symmetrical and asymmetrical may be used herein within the broad scope of the present invention.

It will be further appreciated by those skilled in the art that cage 12 and filter 14 are intended to be radially compressible and can be radially self-expanding to be delivered to a selected site in compressed form in a delivery catheter or the like and then allowed to simultaneously expand radially. Thus, cage 12 and filter 14 can be made of a resilient material which can be compressed to a smaller diameter and cylindrical form for insertion into the lumen of a delivery catheter and then allowed to expand radially to the form illustrated in the figures for use in a vessel or body passage. Alternatively, cage 12 can be compressed, inserted into a delivery catheter and delivered to the desired site in a blood vessel or the like and expanded therein either by self-expansion or by means of a balloon catheter. It is contemplated that filter 14 can be compressed in a delivery catheter and subsequently inserted into cage 12 in the vessel and allowed to self-expand therein.

Cage 12 and filter 14 can be made of any suitable material using a variety of methods. Suitable conventional materials and methods are well known in the art. Nitinol and stainless steel are examples of suitable materials but other materials may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter and reduction in diameter when withdrawn into the lumen of the retrieval catheter. Thus, the material must also be sufficiently resilient to accomplish the compression and expansion required for use of filter 10.

Suitable methods of manufacture of cage 12 and filter 14 include cutting a pattern into a tube to enable expansion of the tube into the desired main structure and optionally arms as well. Another suitable method is forming the main structure and arms from separate strips or wires and then joining the respective parts together by suitable methods which are well known in the art. Of course, it will be appreciated by those skilled in the art that the main structure can have many different designs and may be modified within the spirit and scope of the present invention. For example, the main structure can have additional struts and can be symmetrical or asymmetrical in form.

Having described a medical filter of the present invention, further understanding of the unique character and advantages of the present invention will be had by the following description of its intended use. Cage 12 and filter 14 are separate pieces which are used in mechanically interfering relationship. Thus, cage 12 and filter 14 can be assembled by pushing filter 14 axially past ribs 22 or ribs 24 depending upon the direction of insertion and into the hollow interior 16 of cage 12 and then both cage 12 and filter 14 can be radially compressed and inserted into a delivery catheter. Then filter system 10 comprising cage 12 and filter 14 can be delivered to a blood vessel or the like and allowed to simultaneously expand in the vessel. Alternatively, cage 12 can be compressed, inserted into a delivery catheter and delivered to the desired site in a blood vessel or the like and expanded therein. Filter 14 can be compressed in a delivery catheter and subsequently inserted into cage 12 in the vessel by pushing filter 14 axially past either ribs 22 or 24 depending upon the direction of insertion into hollow interior 16 of cage 12 and allowed to expand therein. Filter 13 can be pushed by a push wire or mandrel preferably carried in a catheter.

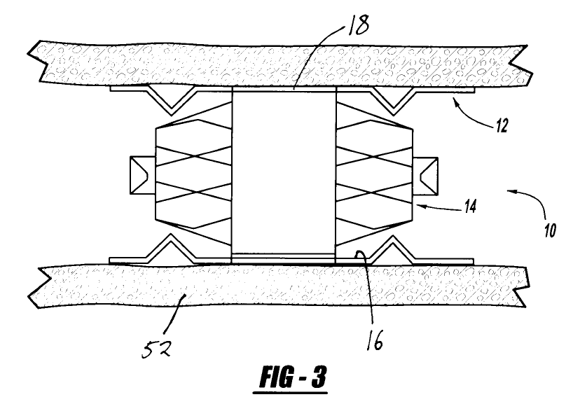

FIG. 3 illustrates medical filter system 10 expanded and in use in a blood vessel 52. Cage 12 is firmly secured in place in vessel 52 by mechanical interference between its wire mesh 18 and the vessel wall. Of course, after insertion, cage 12 will be further secured in vessel wall by cell growth of the wall over the wire mesh structure of cage 12. Optionally, cage 12 can have barbs or other further mechanical attachment devises to further secure cage 12 in vessel 52.

Since the structures of both cage 12 and filter 14 can be well defined during manufacture to complement each other, it will be appreciated that filter 14 can be optimally positioned in vessel 52 by cage 12. Furthermore filter 14 is prevented from unwanted axial migration in the vessel by rib 22 and/or rib 24 which act to mechanically interfere with axial movement of filter 14 leftwardly and rightwardly respectively as viewed in FIG. 3. Should filter 14 tend to move leftwardly, filter elements 42 contact rib 24 while rightward movement of filter 14 is opposed when filter elements 34 come into contact with rib 22. Of course, it will be appreciated by those skilled in the art that, depending upon the specific design of filter 14, it may not be necessary to have ribs 22 and 24 which extend entirely around the circumference of cage 12, but tabs or other radially inwardly extending elements can be substituted for ribs 22 and 24 so long as they come into interfering relationship with filter 14 to prevent unwanted axial migration thereof. Of course, it will also be appreciated by those skilled in the art that filter 14 is resilient and flexible and can be pulled past ribs 22 and 24 by the relatively strong pulling force that can be exerted by a snaring wire of a retrieval catheter. Thus, ribs 22 and 24 serve to prevent unwanted migration of filter 14 but do not substantially interfere with the retrieval of filter 14. It will also be appreciated that cage 12 serves to space filter 14 from blood vessel 52 to minimize cell growth thereover which would interfere with retrieval of filter 14 after it has been it has been in place for an extended duration (more than two weeks). Furthermore, should it be desired, cage 12 can be provided with radially inwardly extending projections or other spacers to further increase the distance filter 14 is spaced from blood vessel 52.

Once emplaced in a body passage such as a vein, in many cases there will be no desire to retrieve filter 14 from the vessel. However, in some cases there will be a desire to retrieve filter 14 from the vessel and in such cases the present invention provides a filter system 10 which facilitates retrieval of filter 14 since there is minimal tissue growth over structural parts of filter 14. Cage 12 provides optimal orientation of filter 14 and stands off filter 14 from vessel 52 to keep it the structure of filter 14 away from post-treatment proliferative tissue growth.

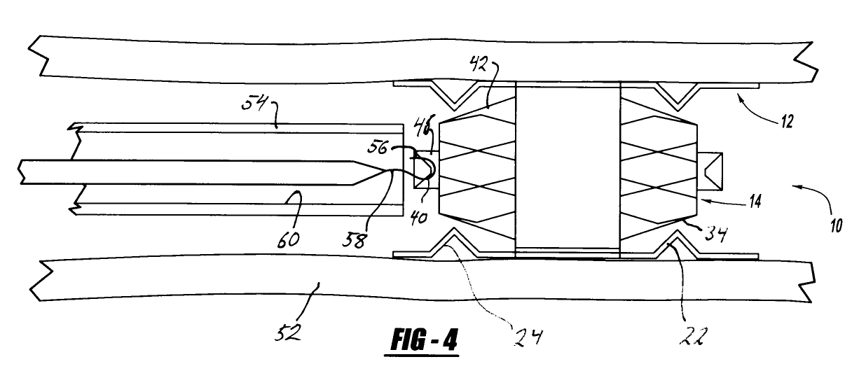

Now referring to FIG. 4, retrieval of filter 10 from blood vessel 52 is illustrated. As illustrated in FIG. 4, a retrieval catheter 54 is shown having been positioned proximate to filter system 10. Snare 56 of wire 58 has been hooked onto retrieval hook 40. Filter 14 can now be retrieved by pulling wire 58 leftwardly as viewed in FIG. 4 which will compress filter elements 42 slightly to allow filter 14 to be pulled leftwardly out of cage 12 and into lumen 60 of retrieval catheter 54. Where filter 14 is provided with retrieval hooks at both longitudinal ends thereof, it may be removed from either direction. Thus, filter system 10 may be inserted from either the femoral or cranial direction and filter 12 may be removed from either direction so long as appropriate retrieval hooks are provided for snaring. It will also be appreciated that only one rib 22 or 24 is necessary if it is required to anchor filter 14 from axial movement in only one direction.

While preferred embodiments of the present invention have been specifically described above, it will be appreciated by those skilled in the art that the present invention is subject to variations and modifications. For example, main structure 12 can be modified to have different designs within the scope of this invention and ski barbs 14 can be modified to have different geometric shapes within the scope of this invention which is intended to be limited only by the following claims.

What is claimed is:

1. A medical filter system for placement in a tubular body passage such as a blood vessel, the medical filter system comprising in radially expanded form, an elongated cage and an elongated filter, said cage having a tubular form with a hollow interior and said filter disposed within said hollow interior in an interference fit with said cage.

2. A filter system as set forth in claim 1, wherein said filter has a retrieval hook at a longitudinal end thereof.

3. A filter system as set forth in claim 1, wherein said cage is a stent.

4. A filter system as set forth in claim 1, wherein said cage has a reduced diameter portion which interferes with movement of said filter in at least one axial direction.

5. A filter system as set forth in claim 1, wherein said cage has at least one element extending radially inwardly to interfere with movement of said filter in at least one axial direction.

6. A filter system as set forth in claim 1, wherein said cage has spacer elements extending radially inwardly therefrom to stand of said filter radially inwardly from said cage.

7. A filter system as set forth in claim 1, wherein said cage has barbs adapted to secure said cage to a wall of an associated body passage.

8. A filter system as set forth in claim 1 wherein said system is comprised of a material selected from the group consisting of nitinol and stainless steel.

9. A filter system as set forth in claim 1, wherein filter has a retrieval hook at each longitudinal end thereof.

10. A filter system as set forth in claim 1 wherein said cage is self-expanding with a pre-formed tubular expanded shape and said filter is self-expanding with a preformed expanded shape, said filter having a first filter basket and a second filter basket connected to each other by a plurality of longitudinally extending parallel struts, and each longitudinal end of said filter having a retrieval hook thereon.

11. In combination, a blood vessel having a tubular wall and a medical filter system, said medical filter system being expanded in said blood vessel and having an elongated cage and an elongated filter, said cage having a tubular form with a hollow interior and said filter disposed within said hollow interior in an interference fit with said cage.

12. The combination as set forth in claim 12, wherein said filter has a retrieval hook at a longitudinal end thereof.

13. The combination as set forth in claim 12, wherein said cage has a reduced diameter portion which interferes with movement of said filter in at least one axial direction.

14. The combination as set forth in claim 12, wherein said cage has at least one element extending radially inwardly to interfere with movement of said filter in at least one axial direction.

15. The combination as set forth in claim 11, wherein said cage has spacer elements extending radially inwardly therefrom to stand of said filter radially inwardly from said cage.

16. A method of treatment of a patient having a tubular blood vessel with a wall, said method comprising implanting a medical filter system in a tubular body passage, said medical filter system having self-expanding tubular cage containing a self-expanding filter.

17. A method of treatment as set forth in claim 16, wherein said cage has a tubular form with a hollow interior and said filter is disposed within said hollow interior in an interference fit with said cage and said filter has a retrieval hook at a longitudinal end thereof.

18. A method of treatment as recited in claim 16, wherein said cage has a reduced diameter portion which interferes with movement of said filter in at least one axial direction.

19. A method of treatment as recited in claim 16 wherein said cage is implanted in said passage and then said filter is released and allowed to expand in said cage.

20. A method of treatment as recited in claim 16 wherein said cage and said filter are implanted and allowed to expand simultaneously in said passage.
